(19) United States
(12) Patent Application Publication
(10) Pub. No.: US 2018/0258485 A1
(43) Pub. Date: Sep. 13, 2018

(54) Boranephosphonate Detection Probes and Methods For Producing and Using the Same

(71) Applicant: The Regents of the University of Colorado, a body corporate, Denver, CO (US)

(72) Inventors: Subhadeep Roy, Lafayette, CO (US); Marvin Caruthers, Boulder, CO (US); Rajen Kundu, Boulder, CO (US)

(73) Assignee: The Regents of the University of Colorado, a body corporate, Denver, CO (US)

(21) Appl. No.: 15/914,602

(22) Filed: Mar. 7, 2018

Related U.S. Application Data
(60) Provisional application No. 62/468,032, filed on Mar. 7, 2017.

(51) Int. Cl. C12Q 1/6876

(52) U.S. Cl. CPC C12Q 1/6876

(57) ABSTRACT

The present invention provides a detection probe and a method for using and producing the same. The detection probe of the invention comprises boranephosphonate moiety and a target selective moiety. The present invention also provides a method for using the metal ion reducing properties of boranephosphonates (BPs) to determine the presence, the concentration or the location of a target molecule in a sample.

### CROSS-REFERENCE TO RELATED APPLICATIONS

This application claims the priority benefit of U.S. Provisional Application No. 62/468,032, filed Mar. 7, 2017, which is incorporated herein by reference in its entirety.

### FIELD OF THE INVENTION

The present invention relates to a detection probe comprising boranephosphonate moiety and a target selective moiety. The present invention also relates to a method for using and producing the same. In particular, the present invention relates to using the metal ion reducing properties of boranephosphonates (BPs) to determine the presence, the concentration or the location of a target molecule in a sample.

### BACKGROUND OF THE INVENTION

There are a variety of chemical detection probes or sensors to determine the presence of a target molecule in a sample. Often these detection probes or sensors utilize a fluorescence moiety, or other means of allowing detection using various instruments such as an electron microscope, a UV/Vis instrument, an infrared (“IR”) instrument, nuclear magnetic resonance (“NMR”) instruments, etc.

Metallic nanoparticles (MNPs) of noble metals such as Ag and Au posses unique optical, electronic and chemical properties making them widely useful for sensors, probes and diagnostics. They allow sensitive detection using a number of modalities such as electron microscopy, optical microscopy, light scattering, absorbance, fluorescence and by simple visual means, i.e., without the aid of any spectrometric instrument. Method can also include taking a photograph (e.g., using a digital photographic equipment of non-digital photographic equipment) and analyzing the photograph (e.g., using a computer software) to determine the presence or the location of or the quantification of the target molecule.

However, MNPs are typically 5-100 nm in diameter, which is significantly larger than the chemical detection probes or sensors to which they are attached. This leads to problems such as modifications of the binding characteristics of the sensor/probe, unintended binding interactions mediated by the MNP itself and lack of accessibility to the required sites in cells or tissues. Moreover, many MNP-probe conjugates are unstable to conditions such as high salt concentrations and elevated temperatures and cannot be dried, thereby creating difficulties in handling and transport.

Therefore, there is a need for a method that can utilize the advantages of MNPs, such as sensitivity and a wide variety of detection methods offered by MNPs without the traditional drawbacks resulting from conjugation of large MNPs to probes and sensors.

### SUMMARY OF THE INVENTION

Some aspects of the invention are based on the metal ion reducing properties of boranephosphonates (BPs). In general, BP is stable, can be used as a small-molecule tag and has minimal effects on the sensor/probe to which it is appended. In one particular embodiment, BP containing detectors/probes are designed such that treatment with metal ions after binding to the target analyte leads to in situ production of MNPs. Thus, boranephosphonate detection probes/sensors of the invention offer the advantages of MNPs (e.g., sensitivity and multi-modal detection) without the traditional drawbacks resulting from conjugation of large MNPs to probes and sensors.

Other aspects of the invention provide methods for producing and using the boranephosphonate detection probes/sensors. In some embodiments, methods for using boranephosphonate probes take advantage of the metal ion reducing properties of boranephosphonate group to produce metal nanoparticles (MNPs), which is then detected using various methods that are available for determining the presence of MNPs, such as electron microscopy, optical microscopy, light scattering, spectrometric methods (e.g., absorbance, fluorescence, etc.) as well as simple visual means, and other analytical methods known to one skilled in the art.

One particular aspect of the invention provides a detection probe comprising a boranephosphonate probe moiety and a target selective moiety. The boranephosphonate probe moiety can optionally be linked to the target selective moiety. In some embodiments, the boranephosphonate probe moiety is used as a detection probe to indicate the presence, absence or a location of a target molecule. Yet in other embodiments, the target selective moiety is used as a binding moiety to form a target molecule-target selective moiety complex when the target molecule is present in a sample that is analyzed using the method of the invention described herein.

In one particular embodiment, the detection probe is a molecule of the formula:

Q¹-(N¹)ₓ—(N²-bp)_{y}-(Q²)_{z}

where
x is an integer from 0 to 500, typically from 1 to 400, often from 5 to 200, more often from about 10 to about 100, and still more often from about 10 to about 50;
y is an integer from 1 to 50, typically form 1 to about 40, often from 1 to about 30, and more often from about 3 to 20;
z is an integer from 1 to 50, typically from about 1 to 40, often from 1 to about 30, more often from about 1 to about 20, and still more often from about 1 to about 10;
bp is boranephosphonate moiety (e.g., a moiety of the formula (H₃B⁻)—P(═O)—(O−)₂);
each of N¹ and N² is independently a nucleotide or an analog thereof;
Q¹ is a probe, a nucleotide or an analog thereof; and
Q² is a nucleotide or an analog thereof.

Yet in some embodiments, the detection probe comprises a plurality of said boranephosphonate probe moieties. Typically, the detection probe includes from 1 to about 50, typically from 1 to about 40, often from 1 to about 30, and more often from about 3 to about 30, and still more often from about 3 to about 20 boranephosphonate probe moieties. In another embodiment, the detection probe comprises from 1 to about 20 boranephosphonate probe moieties.

Still in other embodiments, the target selective moiety comprises an oligonucleotide moiety.

In other embodiments, said target selective moiety comprises a CRISPR-cas9 system having a small guide RNA oligomer (sgRNA) and a cas9 variant lacking active endonuclease domains (dcas9). In some instances the sgRNA includes or is linked to the boranephosphonate probe moiety. It should be appreciated that in such instances, the boranephosphonate probe moiety can be located within the sgRNA that electively binds to the target genomic sequence or it can be attached or linked to the sgRNA and be separate from the selective binding portion of the sgRNA.

Still yet in other embodiments, said target selective moiety comprises a DNA intercalator. In this instance, the boranephosphonate probe moiety can be linked or attached to the DNA intercalator directed or optionally by a linker.

The boranephosphonate probe moiety can be attached or linked to the target selective moiety optionally through a linker or the boranephosphonate probe moiety and the target selective moiety can be two separate molecules.

Another aspect of the invention provides a method for detecting the presence or the location of a target molecule in a sample using the detection probe disclosed herein. In one embodiment, the method includes:
contacting a sample with a detection probe disclosed herein under conditions sufficient to form a probe-target complex when a target molecule is present in the sample;
reacting said boranephosphonate probe moiety with a metal ion solution under conditions sufficient to produce a metal nanoparticle (MNP); and
analyzing the MNP to determine the presence of or the location of said target molecule in said sample.

In some embodiments of the method disclosed herein, analysis of the MNP is conducted by visualization, using an electron microscopy, using a light microscopy, using a spectrometer, or a combination thereof. Still in other embodiments, the target molecule is a nucleic acid sequence.

Yet in other embodiments, the sample comprises a cell, a chromatin or a tissue section. In such embodiments, the method can be used to determine the location of a target nucleic acid sequence within the chromatin. Still in other embodiments, said detection probe comprises a CRISP-cas9 system having a small guide RNA oligomer (sgRNA) and a cas9 variant lacking active endonuclease domains (dcas9). In some instances, the sgRNA comprises or is attached to said boranephosphonate probe moiety.

In other embodiments of the methods disclosed herein, said target selective moiety comprises a DNA intercalator. In such embodiments, in some instances said boranephosphonate probe moiety is linked to said DNA intercalator.

Still further, in some embodiments said target selective moiety is attached to a surface of a solid substrate that is capable of selectively binding to a portion of said target molecule to form a target-capture hybrid complex having a portion of said target molecule that is unbound to said target selective moiety when said target molecule is present in said sample, and wherein said boranephosphonate probe moiety is capable of binding to at least a portion of said unbound portion of said target molecule.

### BRIEF DESCRIPTION OF THE DRAWINGS

FIG. 1 shows examples of some of the BP containing small molecules of the present invention (compounds A-C) that can be covalently attached to different sensors and probes. Compounds D-F are examples of BP-containing DNA intercalators of the present invention that can be used in assays.

FIG. 2 is a schematic illustration of one particular method of the invention for producing small molecules with boranephosphonate moieties.

FIG. 3 shows some of the representative small molecule detection probes of the invention.

FIG. 4 is a schematic illustration of a method of the invention for producing a DNA/RNA intercalator with boranephosphonate moiety.

FIG. 5 shows photographs of a fluorescein labeled, bpT₂₁ oligomer (A, green channel) that is internalized by HeLa cells and concentrated in endosomal vesicles. DAPI stain in (A) is shown in blue. Photographs (B) and (C) are TEM image of bpT21 treated cells showing specific gold NP deposition in endosomal vesicles. In these uranyl acetate stained sections, the gold NPs appear as black dots and the endosomes shown as low contrast spherical or ovoid structures. The sale bars are 50 nm.

FIG. 6 shows photographs of in situ hybridization using a BP containing antitelomere probe (Cy5-[CCCTAA]₆-[T⋅T]₂; ⋅=BP) on Tokuyasu type cryosections of mitotic U2OS cells, followed by treatment with gold ion solution led to selective deposition of metal nanoparticles (NPs) at discreet spots on the chromatin. The scale bar corresponds to 2 μm. In a separate experiment, co-labeling with the same Cy5 containing anti-Telomere probe and an anti-TIRF2 antibody conjugated to Alexa 488 dye on the same type of cryosections and visualized by fluorescene microscopy showed exact overlap denoting specificity of binding of the probe of the present invention to telomeres. Photograph (B). The cellular DNA was stained by DAPI.

FIG. 7 shows a schematic illustration (left panel or panel A) of Telo-BP-sgRNA that was produced and used in CRISPR-cas9 probe Example. The Cy3 signal from the BP-sgRNA is shown in red and the staining of the nucleus by the live cell penetrating dye Hoechst 33258 is shown in blue (panel B).

FIG. 8 is confocal laser scanning microscopy images of cell sections with a cytoplasmic and nuclear Cy3 signal, panels A and D, respectively; TEM images, panels B and E, of the same sections, respectively; and higher magnification images corresponding to the regions shown in dashed black boxes, panels C and F.

FIG. 9 is a schematic presentation and layout of the slide used in assay experiments.

FIG. 10 is images and the pixel quantification of the gold deposited slide of DNA binding. The quantification results represent an average pixel intensities obtained from three individual experiments.

FIG. 11 is images and the pixel quantification of the gold deposited slide of RNA binding. The quantification results represent an average pixel intensities obtained from three individual experiments.

FIG. 12 shows melting curve of target DNA with detection probes containing varying numbers of boranephosphonate linked oligodeoxythymidine tags (BP-5, BP-10, BP-20 and BP-30).

FIG. 13 shows melting curve of target RNA with detection probes containing varying numbers of boranephosphonate linked oligodeoxythymidine tags (BP-5, BP-10, BP-20 and BP-30).

### DETAILED DESCRIPTION OF THE INVENTION

The present invention provides detectors/sensors that are useful in determining the presence of or the location of a target molecule in a sample. In general, unless the context requires otherwise, the terms “detector,” “detector probe,” “detection probe,” “probe,” and “sensor” when referring to a chemical compound are used interchangeably herein and refer to a chemical compound that is used to determine the presence of or the location of a target molecule.

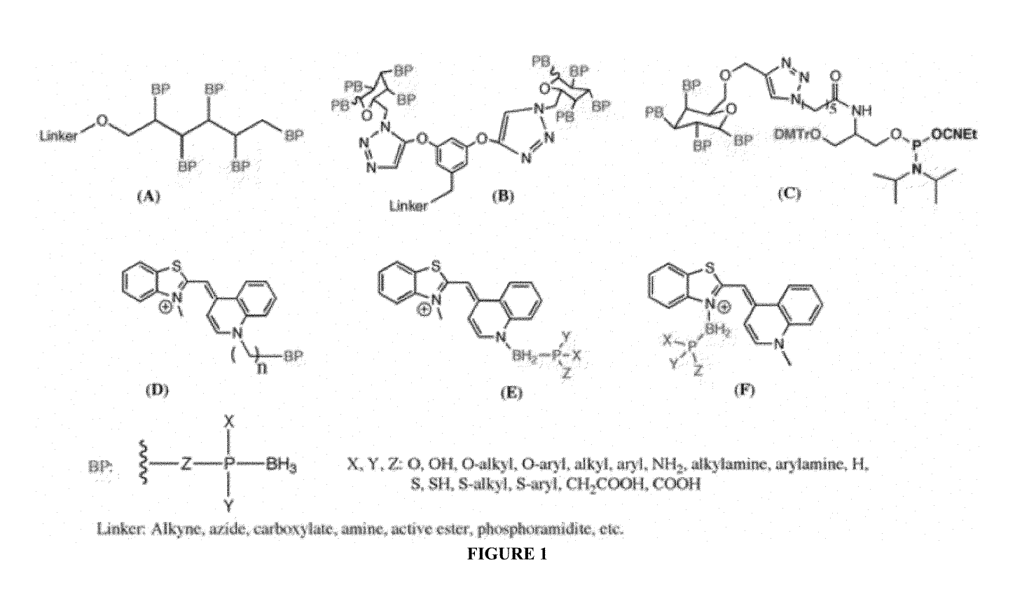

Detection probes of the invention include a boranephosphonate moiety and a target selective moiety. In one particular embodiment, boranephosphonate moiety is boranephosphonate-pyridinium, e.g., (Z)(X)(Y)P—BH₂-Pyr (where Pyr=pyridine, and a target selective moiety is attached to Z, for X, Y and Z, see, for example, FIG. 1). Boranephosphonates (BP) are a class of phosphate derivatives that contain a borane (BH₃ or —BH₂—) group coordinated to the phosphorous atom (see, for example, FIG. 1). BP groups are stable to ambient conditions but upon exposure to metal ions (e.g., Ag+, Au+, Au3+, Pt²⁺, Pd²⁺, etc.) they reduce the metal ions and produce metallic nanoparticles (HNPs). MNPs of noble metals such as Ag and Au possess unique optical, electronic and chemical properties making them widely useful for sensors, probes and diagnostics. They allow sensitive detection using a number of modalities such as electron microscopy, optical microscopy, light scattering, absorbance, fluorescence as well as by visual means.

In some embodiments, the boranephosphonate probe moiety comprises an oligonucleotide linked via boranephosphonate internucleotide linkages. Still in other embodiments, the boranephosphonate probe moiety is of the formula:

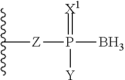

where X¹ is O or S; Y is linked to a riboside moiety of a nucleotide, H or —R^{a} (where R^{a} is alkyl or aryl), —X²R^{b} (where X² is O or S, and R^{b} is H, alkyl or aryl), —NR^{c}R^{d} (where each R^{c} and R^{d} is independently H, alkyl, or aryl), CH₂COOH, or COOH; and Z is R^{e} (where R^{e} is alkylene or arylene), —X²R^{e}, —NR^{c}R^{f} (where R^{f} is a bond or R^{e}), —R^{g}COOH (where R^{g} is a bond, i.e., absent, or alkylene), or riboside moiety of a nucleotide.

Still in other embodiments, the boranephosphonate probe moiety and the target selective moiety are part of a same molecule. For example, an oligonucleotide containing BP internucleotide linkages that can bind to its complementary sequence or an aptamer containing BP linkages that can both bind to the target molecule and produce metal nanoparticles.

Another aspect of the invention provides a diagnostic kit comprising a solid substrate and a boranephosphonate probe molecule. The solid substrate includes a surface bound target selective binding molecule. In this manner, the solid substrate is used to bind to at least a portion of the target molecule, if present in a sample, to form a target selective binding molecule-target molecule complex. The boranephosphonate probe molecule binds to a portion of the target molecule that is not bound to the target selective binding molecule. This allows a “sandwich-like” assay to be performed.

The target selective moiety attaches to a desired target molecule, if present, and the boranephosphonate (“BP”) moiety is used to reduce metal ions to produce MNPs. Detection of MNPs using any of the conventional methods then allows determination of the presence of or the location of a desired target molecule. Target selective moiety can be a small molecule (e.g., a ligand for a receptor, enzyme, a snap-tag or Halo-tag substrate, etc.), DNA and/or RNA intercalator, a peptide, a protein, an aptamer, an oligonucleotide, DNA or RNA minor groove binder, DNA/RNA major groove binder, G-quadruplex binders, etc. Unless context requires otherwise, the terms “nucleic acid” “polynucleotide” and “oligonucleotide” are used interchangeably herein and refer to a deoxyribonucleotide or ribonucleotide polymer in either single- or double-stranded form, and unless otherwise limited, encompasses known analogs (i.e., derivatives) of natural nucleotides that hybridize to nucleic acids in a manner similar to naturally-occurring nucleotides. Examples of such analogs or derivatives include, without limitation, phosphorothioates, phosphoramidates, methyl phosphonates, chiral-methyl phosphonates, alkylated and protected ribonucleotides (e.g., 2-O-methyl ribonucleotides, acetonated ribonucleotides, acetylated ribonucleotides, etc.), and peptide-nucleic acids (PNAs). Typically, an oligonucleotide has from about 2 to about 500, often from about 5 to about 200, more often from about 10 to about 100, and most often from about 10 to about 50 nucleic acids. The term “about” when referring to a numeric value means±20%, typically ±10%, and often ±5% of the stated numeric value.

Some of the examples of detection probes of the invention are illustrated in FIG. 1, where compounds (A)-(C) are examples of small molecule tags or detector probes and compounds (D)-(F) are DNA/RNA intercalators with BPs.

The term “alkyl” refers to a saturated linear monovalent hydrocarbon moiety of one to twelve, typically one to six, carbon atoms or a saturated branched monovalent hydrocarbon moiety of three to twelve, typically three to six, carbon atoms. Exemplary alkyl group include, but are not limited to, methyl, ethyl, n-propyl, 2-propyl, tert-butyl, pentyl, and the like. Alkyl can be optionally substituted with halogen, alkoxide (e.g., —OR′, where R′ is alkyl), etc. “Alkylene” refers to a saturated linear saturated divalent hydrocarbon moiety of one to twelve, preferably one to six, carbon atoms or a branched saturated divalent hydrocarbon moiety of three to twelve, preferably three to six, carbon atoms. Exemplary alkylene groups include, but are not limited to, methylene, ethylene, propylene, butylene, pentylene, and the like. The term “aryl” refers to a monovalent mono-, bi- or tricyclic aromatic hydrocarbon moiety of 6 to 15 ring atoms which is optionally substituted with one or more, typically one, two, or three substituents within the ring structure such as, but not limited to, phenyl, naphthyl, anthracenyl, etc. When two or more substituents are present in an aryl group, each substituent is independently selected. Exemplary substituents for an aryl group include halide (F, Cl, Br and I), alkyl, alkoxide, nitro, cyano, etc. “Arylene” refers to a divalent aryl as defined herein. Exemplary arylene groups include, but are not limited to, phenylene, naphthylene, anthracenylene, and the like. The term “aptamer” (i.e., nucleic acid antibody) is used herein to refer to a single- or double-stranded DNA or a single-stranded RNA molecule that recognizes and binds to a desired target molecule by virtue of its shape. See, for example, PCT Publication Nos. WO92/14843, WO91/19813, and WO92/05285, the disclosures of which are incorporated by reference herein.

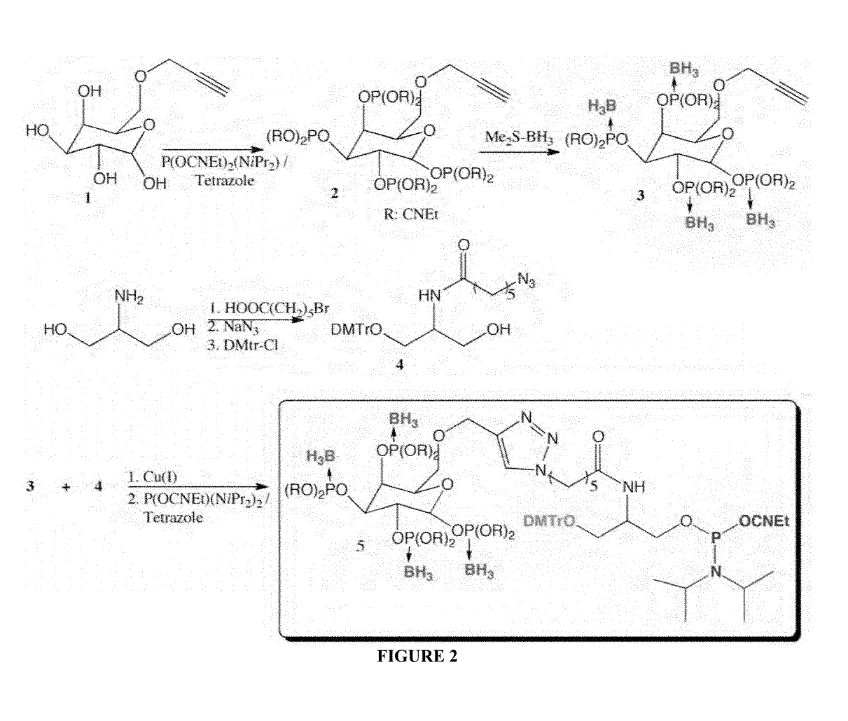

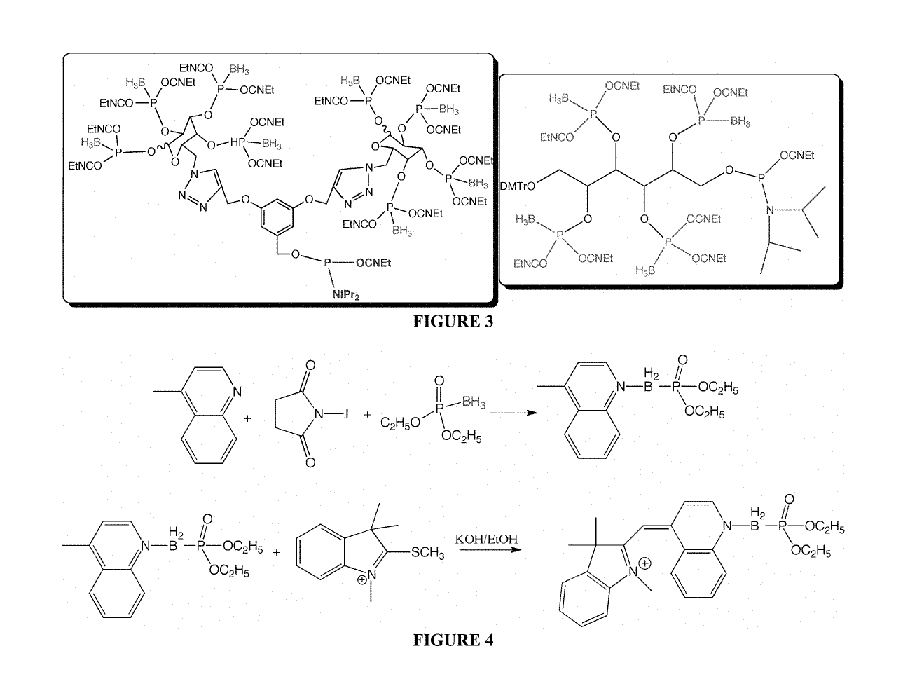

Compounds of the invention can be prepared using conventional methods. See, for example, H. McCuen et al., J. Am. Chem. Soc., 2006, 128, 8138-8139; S. Roy et al., J. Am. Chem. Soc., 2013, 135, 6234-6241; H. Krishna et al., J. Am. Chem. Soc., 2011, 133, 9844-9854; Sergueev, D. S.; Shaw, B. R. J. Am. Chem. Soc. 1998, 120, 9417-9727; Higson, A. P.; Sierzchala, A.; Brummel, H.; Zhao, Z.; Caruthers, M. H. Tet. Lett 1998, 39, 3899-3902; Zhang, J.; Terhorst, T.; Matteucci, M. D., Tet. Lett. 1997, 38, 4957-4960; Shimizu, M.; Saigo, K.; Wada, T., J. Org. Chem. 2006, 71, 4262-4269, all of which are incorporated herein by reference in their entirety. Exemplary synthetic methods that can be used to prepare detection probes of the invention are illustrated in FIGS. 2 and 4. FIG. 3 shows other small molecule detection probes of the invention. It should be appreciated FIGS. 2 and 4 are provided solely for the purpose of illustrating how to prepare some of the detection probes of the invention and do not constitute limitations on the scope thereof. One skilled in the art having read the present disclosure can readily prepare other detection probes containing one or more boranephosphonate functional groups.

Briefly, as described in J. Am. Chem. Soc., 2013, 135, 6234-6241, automated bpDNA synthesis can be carried out on an ABI 394 Synthesizer. In one particular example, syntheses of bpDNA were performed at a 0.2 μmol scale using a 5′-DMT 2′-deoxythymidine joined to a low volume polystyrene solid support via a succinate linkage. For synthesis of 2′-deoxyoligonucleotides, a standard 0.2 μmole synthesis cycle was used with an increased coupling time of 120 s. A wash with methanol following the detritylation step was added. Starting materials (e.g., commercially obtained 5′-O-DMT-2′-deoxythymidine 3′-O-methyl N,N-diisopropylaminophosphoramidite (Glen Research)) were dissolved in anhydrous CH₃CN and the reagent was dissolved in CH₂Cl₂ at a concentration of 0.1 M. Detritylation was carried out using a 0.5% solution of TFA in anhydrous CHCl₃ that also contained 10% TMPB. Solutions for boronation (0.05 M BH₃.THF complex in THF) and oxidation (1.0 M t-BuOOH in CH₂Cl₂) were prepared fresh prior to use. Reagents for activation (ethylthiotetrazole) and capping were purchased form Glen Research. A stepwise description of the synthesis cycle is well known (e.g., see Table S1 in J. Am. Chem. Soc., 2013, 135, 6234-6241). Deprotection was carried out in two steps: The solid support linked 2′-deoxyoligonucleotides were first treated with a 1.0 M solution of disodium-2-carbamoyl-2-cyanoethylene-1,1-dithiolate in DMF for 1 h followed by extensive washing with DMF and methanol. The resin was then dried using a flow of argon. Subsequently these 2′-deoxyoligonucleotides were desilylated by overnight fluoride treatment (940 μL DMF+470 μL Et3N+630 μL, Et3 N.(HF)3). The resin was washed repeatedly with DMF, Millipore water, and methanol and dried with argon. The resin was then transferred to a glass vial and suspended in 37% ammonia for 1-2 h, and the ammonia was removed by evaporation. The cleaved 2′-deoxyoligonucleotides were dissolved in a 10% acetonitrile-water mixture and used for further analysis and purification.

In one particular embodiment, detectors of the invention use the metal reducing properties of BP groups to produce metal nanoparticles (MNPs) from metallic ions. MNPs are then used as a signal to determine the presence of, concentration of and/or the location of target molecule in a sample. As used herein, “sample” can be a cell, chromatin, a fluid medium, a tissue section, clinical samples (such as blood, saliva, plasma, skin cells, hair, etc.), environmental samples (such as river water, soil sample, etc.), as well as any other biological or environmental samples.

One particular aspect of the invention provides a detection probe comprising a boranephosphonate moiety and a target selective moiety. In some embodiments, the detection probe comprises a plurality of said boranephosphonate moieties. In other embodiments, at least one of the boranephosphonate is boranephosphonate-pyridinium.

Still in other embodiments, the target selective moiety comprises an aptamer, a small molecule (e.g., a drug, a drug candidate, a ligand for a receptor or enzyme, etc.), an oligonucleotide. Yet in some embodiments, the oligonucleotide comprises a deoxyribonucleotide, a ribonucleotide, or a derivative thereof or a combination thereof.

Yet in other embodiments, the oligonucleotide comprises a small guide RNA oligomer (sgRNA). In some instances, the sgRNA has from about 2 to 500, typically from about 5 to about 200, often from about 10 to about 100, and often about 10 to about 50 nucleic acids. Still in some embodiments, the detection probe comprises CRISPR-cas9 system. In some instances, the CRISPR-cas9 system comprises a cas9 variant lacking active endonuclease domains (dcas9). In this manner, the detection probe can be used to selectively bind to chromatin, chromosome, or cells without damage to the sample. CRISPR-cas9 system has been widely used by one skilled in the art to locate or modify a particular gene. For a brief overview of CRISPR-cas9 system, see, for example, Heidi Ledford, Nature, 2016, 531, pp. 156-159 as well as references cited therein, all of which are incorporated herein by reference in their entirety. The terms “small guide RNA” and “guide RNA” are used interchangeably herein and refers to a piece of RNA that consists of a small piece of pre-designed RNA sequence (e.g., from about 10 to about 100 bases long, typically from about 10 to about 50 bases long, often from about 10 to about 40 bases long, more often from about 10 to about 30 bases long, and most often about 20 bases long), typically located within a longer RNA scaffold. The guide RNA ‘guides’ Cas9 (an enzyme) to the desired or right part of the genome. The guide RNA is designed to bind to a specific sequence in the DNA. The guide RNA has RNA bases that are complementary to those of the target DNA sequence in the genome. Thus, the guide RNA will selectively bind to the target sequence of the genome. In one embodiment, cas9 is a variant cas9 lacking active endonuclease domains (dcas9).

In one particular embodiment, the guide RNA can include or be linked to boranephosphonate probe moiety. That is the boranephosphonate moiety can be part of the guide RNA or is a separate moiety that is attached or linked to the guide RNA.

In other embodiments, at least a portion of the oligonucleotide comprises a nucleotide linkage comprising the boranephosphonate. In some instances at least one of the boranephosphonate is boranephosphonate-pyridinium.

Another aspect of the invention provides a method for detecting the presence of, the concentration of, or the location of a target molecule in a sample. The method generally includes:
contacting a sample with a detection probe comprising a boranephosphonate moiety and a target molecule selective moiety under conditions sufficient to form a probe-target complex when a target molecule is present in the sample;
reacting said boranephosphonate moiety with a metal ion solution under conditions sufficient to form a metal nanoparticle (MNP); and
analyzing the MNP to determine whether said target molecule is present or absent in said sample, the concentration of target molecule in the sample or the location of the target molecule in the sample.

Still another aspect of the invention provides a method for identifying the presence, the concentration or the location of a target nucleic acid sequence in a sample. The method includes:
contacting a sample with a detection probe comprising a boranephosphonate moiety and a target nucleic acid selective moiety under conditions sufficient to form a target-probe hybrid complex when said target nucleic acid sequence is present in said sample;
reacting said boranephosphonate moiety with a metal ion under condition sufficient to form a metal nanoparticle (MNP); and
analyzing the MNP to identify the presence, concentration or the location of said target nucleic acid sequence in said sample.

The sample can be a cell, a tissue section, or a chromatin. For example, such a method can be used to determine the location of a target nucleic acid sequence within the chromatin. The method can also be used to determine the presence or the location of a target molecule (e.g., enzyme, receptor, genetic marker, mutation, a particular allele, etc.) in a sample such as a cell or a tissue sample or tissue section.

In some embodiments, the step of contacting the sample with a detection probe comprises:
(i) contacting said sample with a solid substrate comprising a capture probe bound to a surface of said solid substrate under conditions sufficient to form a capture probe-target nucleic acid hybrid complex when said target nucleic acid sequence is present in said sample, wherein said capture probe comprises only a portion of a complementary nucleic acid sequence of said target nucleic acid sequence such that said capture probe-target nucleic acid hybrid complex comprises a hybridized portion and a free target nucleic acid sequence; and
(ii) contacting the resulting solid sample of step (i) with said detection probe under conditions sufficient to form said target-probe hybrid complex when said capture probe-target nucleic acid hybrid complex is present on the surface of said solid substrate, wherein said target nucleic acid selective moiety of said detection probe comprises a complementary nucleic acid sequence of at least a portion of said free target nucleic acid sequence.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

Compounds and methods of the invention can be used to produce MNPs that can be detected by any of the conventional methods for detecting MNPs including, but not limited to, electron microscopy and simple visual means. The following examples illustrate the scope of the invention. However, it should be appreciated that the scope of the invention is not limited to these particular examples.

For these experiments, oligonucleotide probes containing internucleotide boranephosphonate linkages was used. Oligonucleotides having boranephosphonate linkages were readily prepared using method previously disclosed by the present inventors. See, for example, S. Roy et al., J. Am. Chem. Soc., 2013, 135, 6234-6241, which is incorporated herein by reference in its entirety. It should be appreciated other detection probes comprising non-oligonucleotide target selective moieties can be prepared as well. See, for example, FIG. 1.

BP Probes for Electron Microscopy:

Electron microscopy (EM) allows high resolution imaging of biological structures. However, the field of EM lacks effective probes that can label specific cellular molecules or features for their visualization under an electron microscope. BP containing probes offer the ability to label the cellular target with the probe and subsequently upon exposure to metal ions produce EM visible MNPs indicating the location of the targeted cellular feature or molecule. The following are three examples that demonstrates the use of BP probes for these purposes.

Labeling Endosomal Vesicles.

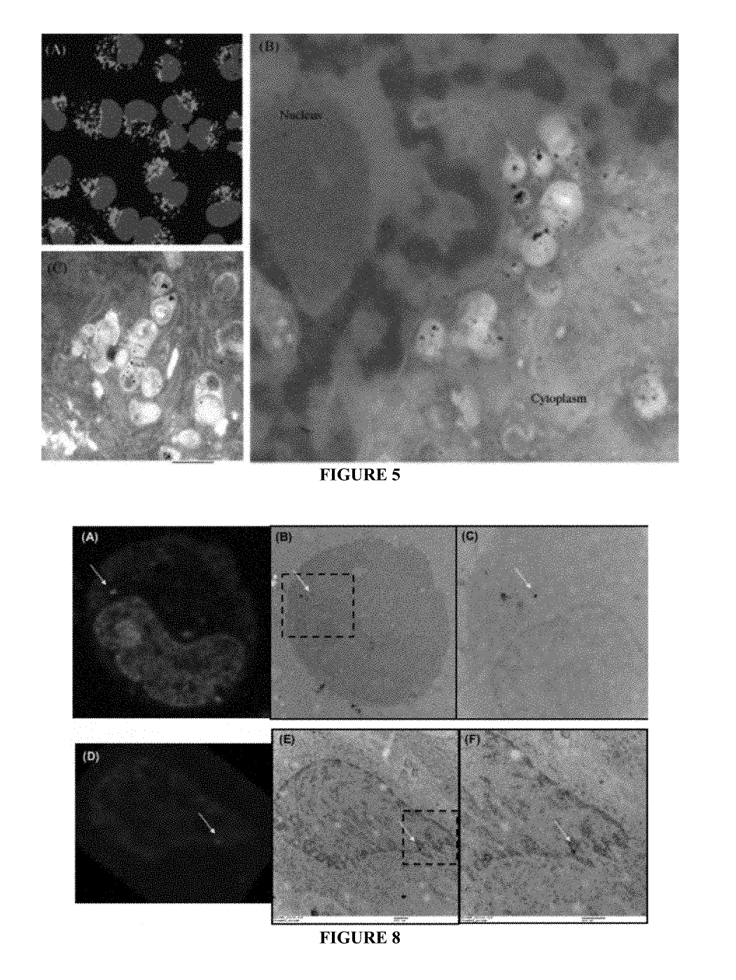

The present inventors have discovered that BP containing DNA oligomers (BP-DNA) were taken up by Hela cells and concentrated in endosomal vesicles (FIG. 5, panel A). Taking advantage of this phenomenon, namely the ability of BP-DNA to specifically label intracellular compartments for EM visualization, the following experiment was conducted.

Hela cells were grown in media containing a fluorescein labeled 21-mer 2′-deoxyoligothymidine BP-DNA sequence containing BP groups at each internucleotide linkage (“BP-dT₂₁”). After washing the cells, imaging by fluorescence microscopy of live cells (FIG. 2, panel A) showed the expected punctate signals arising form entrapment of the BP-dT₂₁ in endosomes. These same cells were then prepared for visualization by EM by high pressure freezing (HPF) followed by freeze substitution (FS) into acetone containing 0.1% glutaraldehyde, chemical fixation by warming to −45° C. and embedding in a K4M lowacryl resin. HPF-FS is a well established procedure that preserves cellular structures and make it possible to observe cells under an electron microscope operating under high vacuum. Thin sections (100 nm) cut from the K4M resin block were then treated with a solution of Gold Enhance EM (Nanoprobes, Inc), post-stained with uranyl acetate and imaged by transmission electron microscopy. The micrographs in FIG. 2 (panels B and C) showed very specific deposition of gold nanoparticles (“NPs”) that was restricted to within endosomes. These experiments demonstrated both the ability to target a specific cellular vesicle as well as the compatibility of BP-DNA mediated EM signaling with the HPF-FS procedure for preparation of cells.

BP-DNA Electron Microscopy In Situ Hybridization Probes.

The present inventors have also successfully labeled the telomeres of mitotic U2OS cells for EM visualization using an anti-telomere in situ hybridization BP-DNA probe, which contained both a boranephosphonate EM-tag and a Cy5 fluorescent label (sequence provided in legend to FIG. 6). The in situ hybridization and treatment with Gold Enhance EM solution was carried out on Tokuyasu type cryosections of chemically fixed and sucrose embedded cells. The electron micrographs revealed gold nanoparticle deposition only at a few discreet sites within the mitotic chromosomes (FIG. 6, panel A). In a separate experiment, the specificity of the probe binding was demonstrated by co-labeling the telomeres of U2OS cells on similar Tokuyasu sections first with an antibody against the telomere binding protein TIRF2 followed by in situ hybridization using BP-DNA probe (FIG. 6, panel B).

BP Containing CRISPR-Cas9 Probes for Labeling Specific Chromosomal Sites for EM Studies.

Structures adopted by chromatin inside the nucleus are both very dense and fragile. For the visualization of the ultrastructural details of these dense structures in three dimensions, the spatial resolution afforded by electron microscopy (EM) remains unparalleled. Unfortunately, the field of EM lacks probes that can label a single specified nucleotide sequence in the context of the entire genome in cells that are preserved in their native state. To overcome this deficiency, the present inventors utilized recent developments in CRISPR gene targeting technology to design BP containing probes that bind their target chromosomal site and produce EM-visible metal nanoparticles.

The CRISPR/cas9 system binds target DNA sequences in living cells and cleaves them using its endonuclease domains. Target specificity is determined by the sequence of a small guide RNA oligomer (sgRNA) with which the cas9 protein forms a complex. In this experiment, a cas9 variant lacking active endonuclease domains (dcas9) and complexed with an sgRNA containing BP groups (“BPsgRNA”) binds the desired target DNA without cleaving. Subsequent fixation, embedding into resins, sectioning and treatment with metal ion solutions produced MNPs to indicate the location of the genomic site of interest for EM studies. The BP-sgRNA was also labeled with fluorescent dyes to enable correlated light and electron microscopy (CLEM). In this scheme as the probes bind their target in live cells, they allowed preservation of the chromatin structure while enabling high resolution EM imaging.

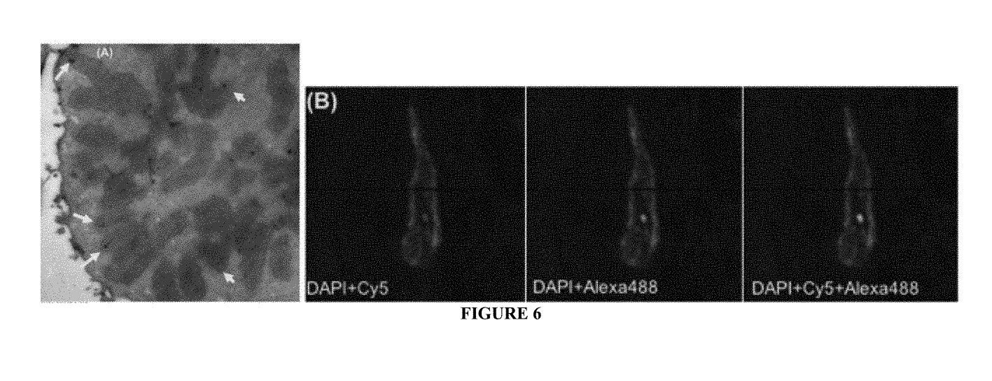

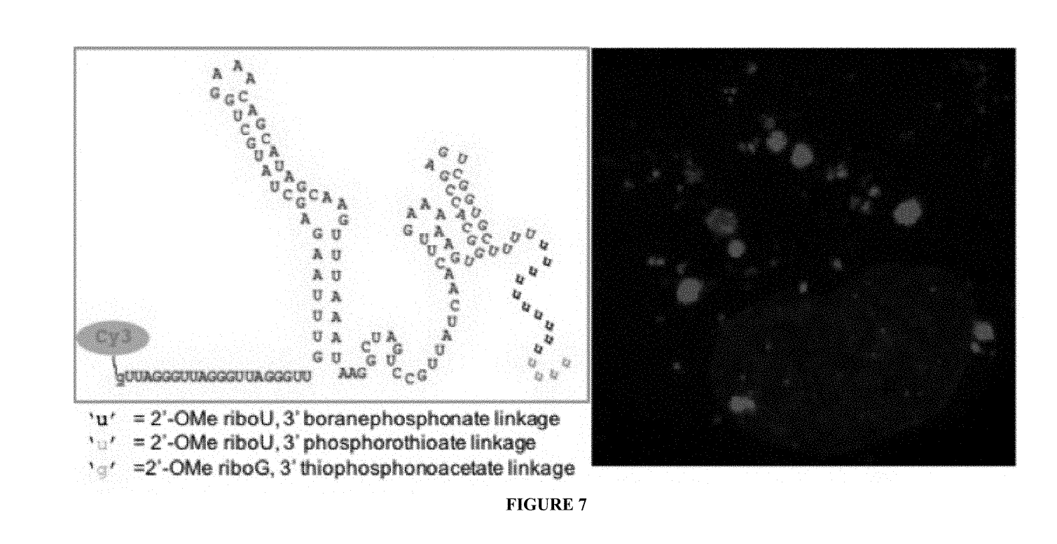

Specifically, a BP containing RNA sequence, called Telo-BP-sgRNA (FIG. 7, panel A) was synthesized. This RNA sequence can form a complex with the dcas9 protein and direct the Telo-BPsgRNA:dcas9 assembly to bind to the telomeric region of mammalian cells. The Telo-BPsgRNA (FIG. 7, panel A) contained the following features: (1) A 3′ segment containing ten BP internucleotide linkages and three phosphorothioate (PS) internucleotide linkages (residues shown in black and blue respectively). The PS linkages bind the MNP produced and prevent their diffusion away from the site of formation; (2) A Cy3 dye attached to the 5′ end, (3) a dcas9 binding segment (shown in green), (4) a target binding site complementary to the mammalian telomeric sequence (shown in red) and (5) a 2′O-Me thiophosphonoacetate linkage at the 5′ terminus (shown in pink) for protection against degradation by 5′ exonucleases.

Clonal RPE cells that stably express dcas9 were transfected with the Telo-BP-sgRNA using the Dharmafect transfection reagent. Fluorescence confocal laser scanning microscopy (CLSM) was carried out on live cells 48 h post transfection. As seen in FIG. 7, panel B, punctate fluorescent signals in the cytoplasm was observed, a typical occurrence from lipid mediated RNA transfection, as well as several smaller puncta in the nucleus indicative of telomeres. Co-immunolabeling with an antibody against TIRF-2, a telomere binding protein, confirmed that the observed nuclear puncta were indeed telomeres (data not shown).

For EM experiments, similarly treated cells were fixed 48 h after the Telo-BP-sgRNA transfection step by high pressure freezing followed by freeze substitution and embedding into Epon resin. Sections (70 nm in thickness) were cut from these resin blocks, mounted on TEM grids and stained with Hoechst 33258 for imaging by CLSM. The same sections were then treated with a solution of Gold Enhance EM (Electron Microscopy Sciences; Hatfield, Pa.) and imaged by transmission electron microscopy. Gold Enhance EM contains gold ions along with an enhancing reagent which enlarges the initial small seed particles produced through reduction of the gold ions by the BP groups. In the CLSM images of these resin embedded cell sections, we were able to observe cytoplasmic and nuclear signals in the Cy3 channel (FIG. 8, panels A and D, respectively). Similarly gold nanoparticles were also observed in the cytoplasm and the nucleus and could be correlated to the corresponding fluroescent images (FIG. 8, panels B, C, E and F).

These BP containing CRISPR/cas9 based EM probes provide several specific advantages including, but not limited to, (1) even though EM provides the highest resolution for studying biological structures there are no competing technologies available that allow labeling of low-copy cellular targets to observe them by EM; (2) deposition of MNPs of gold and silver are particularly attractive for EM as they provide high contrast in electron micrographs and will provide unequivocal indication of the target site; (3) the autocatalytic growth of MNPs provides an inherent signal amplification that translates into probes with high sensitivity; and (4) As demonstrated, incorporation of fluorophores and BP groups can be achieved easily within the same probe and allows straightforward method to carry out correlated light and electron microscopy (CLEM). CLEM allows the marriage of live cell imaging, dynamics and multicolor labeling using LM with the high resolution imaging offered by EM. These dual-modality BP probes makes CLEM possible at the level of a single genomic locus in a single cell.

Design and Synthesis of Detection Probes Containing Boranephosphonate for Detection of Target Sequences Using a Sandwich Assay:

In this example, detection probes were prepared by designing probes where a phosphate diester linked oligonucleotide that was complementary to a part of a target sequence (the binding motif), was conjugated to boranephosphonate linked oligodeoxythymidines of various lengths (the signaling motif). As the signaling motif was not expected to have a significant effect on the recognition of the target, this design allowed testing of the effect of varying the number of boranephosphonate groups on the detection sensitivity, without the confounding effects of decrease in Tₘₛ with increasing numbers of boranephosphonate linkages. Additionally, by positioning the oligothymidines at the 3′ end, the present inventors were able to synthesize the probes using standard, inexpensive phosphoramidites and DNA synthesis reagents with only minor changes to the solid phase oligonucleotide synthesis conditions. The probes also contained a single phosphorothioate linked deoxyoligonucleotide at the 3′ end.

It was believed that binding of the S atoms to the initial metal seed particle produced would prevent loss of signal through diffusion of the seed away from the site of production. Probes containing 5, 10, 20 and 30 BP linkages as the signaling motif were prepared. In each case the binding motif remained identical. For the probes containing 5, 10 and 20 BP linkages the olignucleotides (labeled BP-5, BP-10 and BP-20, respectively) were purified post synthesis using a Glen-Pak cartridge using a DMT-on purification strategy. In contrast, the purification of the BP-30 probe required addition process. Due to the hydrophobic nature of the BP groups present on the 3′ end of the oligonucleotide, both the DMT containing full-length product and the failure sequences adhered to the solid matrix of the Glen-pak column and could not be separated. Purification of this probe was achieved by reverse phase HPLC. Here too the broad nature of the peak due to the diastereomeric nature of the BP linkages led to lower recovery and a lower yield of the pure product when compared to the BP-5, BP-10 and BP-20 probes.

TABLE 1
Oligo sequences used in this study

| Entry | Sequences |
|---|---|
| Capture probe | 5′-NH₂-(CH₂)₆-T₁₅ TCAGTAGGGAGGAAG-GTGGTTAAGTTAATA-3′ (SEQ ID NO: 1) |
| Target | 5′-GGCTCCACTA AATAGACGCA TATTAACTTA ACCACCTTCC |
| DNA/RNA | TCCCTACTGA-3′ (SEQ ID NO: 2) |
| BP-5 | 5′-TGCGTCTATT TAGTGGAGCC T-T-T-T-T-T*T-3′ (SEQ ID NO: 3) |
| BP-10 | 5′-TGCGTCTATT TAGTGGAGCC T-T-T-T-T-T-T-T-T-T-T*T-3′ (SEQ ID NO: 4) |
| BP-20 | 5′-TGCGTCTATT TAGTGGAGCC T-T-T-T-T-T-T-T-T-T-T-T-T-T-T-T-T-T- |
| | T-T-T*T-3′ (SEQ ID NO: 5) |
| BP-30 | 5′-TGCGTCTATT TAGTGGAGCC T-T-T-T-T-T-T-T-T-T-T-T-T-T-T-T-T-T- |
| | T-T-T-T-T-T-T-T-T-T-T-T-T*T-3′ (SEQ ID NO: 6) |

′-′ Boranephosphonate linkage; ′*′ Phosphorothioate linkage

BP Sensors for Visual Detection of Pathogenic Nucleic Acids:

Diagnosis of infections based on the detection of the pathogenic DNA/RNA is the gold standard procedure in resource-rich laboratories. These methods allow high-confidence diagnoses with quantitative measurements, low rates of false results, detection of low-level infections and determination of the subtype of the infecting pathogen. However these tests require sophisticated instruments and centralized laboratories and are ill-suited for resource-poor settings.

Sandwich Assay Using Boranephosphonate Mediated Gold Deposition:

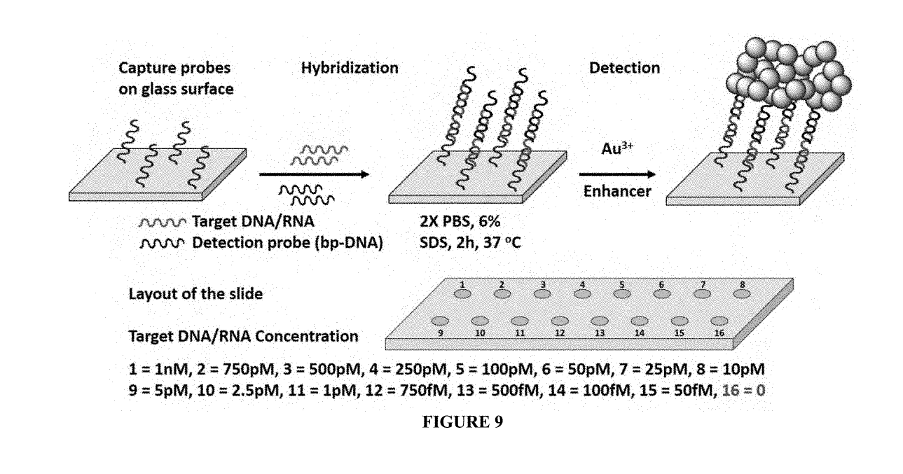

In order to carry out the sandwich assay, a DNA oligonucleotide (capture probe) was covalently attached to the surface of a Code Link glass slide through a terminal primary amino group, using the glass slide manufacturer's protocol at sixteen spots on each slide (FIG. 9). Subsequently a solution containing 0.5 μM of the detection probes and varying amounts of the target sequence (1 nM to 50 fM) were added to the spots and allowed to hybridize for 2 h at room temperature. FIG. 9 is a schematic illustration showing the layout of the slide used in each experiment. The slide was then washed with once with PBS (pH 6.1) for 3 min followed by twice with PBS (pH 7.2) for 3 min each and dried by centrifugation (1 min, 1000 rpm). Finally the spots were treated with GoldEnhanceTMBlots (nanoprobes.com) enhancer solution four times for ten minutes each. The slides were photographed using the camera on Samsung Galaxy S2 smartphone after each treatment. These images were then imported into Image Studio Lite software for quantification of the signals obtained. Separately the spots that could be visually detected at each stage were also noted. For comparison, a single 40 minute treatment with gold enhancer solution instead of the four ten-minute treatment described above was also tested. However, more background signal and a lower detection limit was observed with the single 40 minute treatment.

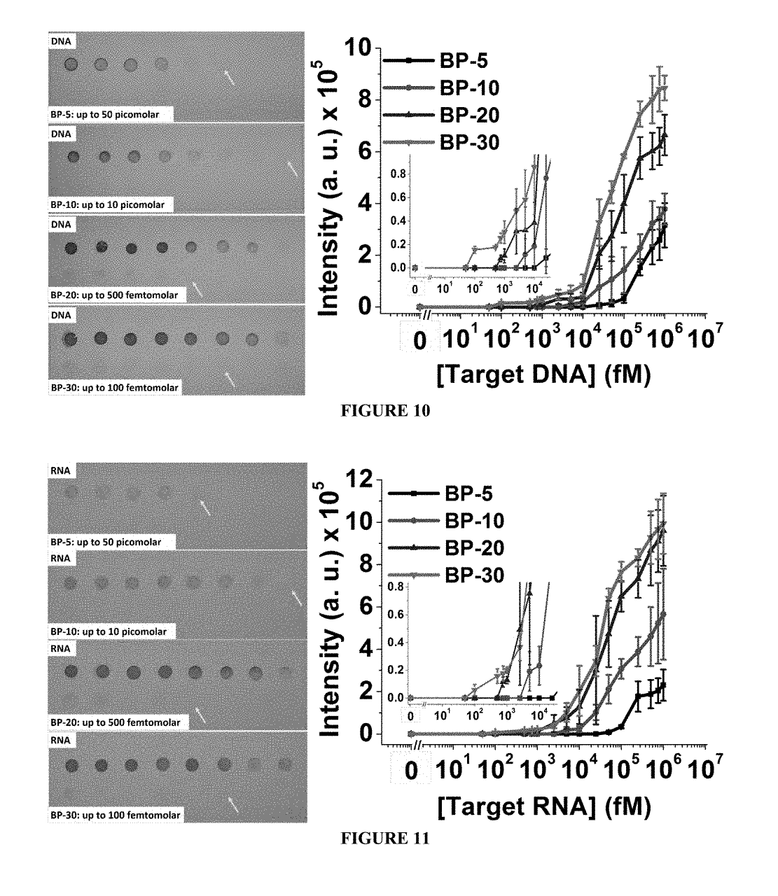

FIG. 10 shows the cell phone camera images as well as the pixel quantification as a function of increasing concentration of the target DNA as well as when using probes with different numbers of boranephosphonate linkages. The picture corresponds to the slide after the last treatment with the gold deposition solution. Increasing the number of BP linkages improved sensitivity of detection. A visual detection limit of 100000 fM, 25000 fM, 1000 fM and 750 fM was obtained when using the BP-5, BP-10, BP-20 and BP-30 probes, respectively. Upon quantification of the density of the spots, the detection limits were found to be 50000 fM, 10000 fM, 500 fM and 100 fM for BP-5, BP-10, BP-20 and BP-30 probes, respectively. The highest sensitivity of 100 fM achieved using the BP-30 probes is comparable to that obtained using gold nanoparticle conjugated DNA probes as reported in the literature. Moreover simply by varying the number of BP linkages or the time of treatment the dynamic range of these probes could be varied over 10⁻¹⁵ orders of magnitude. The same set of experiments was also repeated with an RNA target and similar limits of detection were observed (FIG. 11).

Effect of BP Tag on Binding of Probe to its Target:

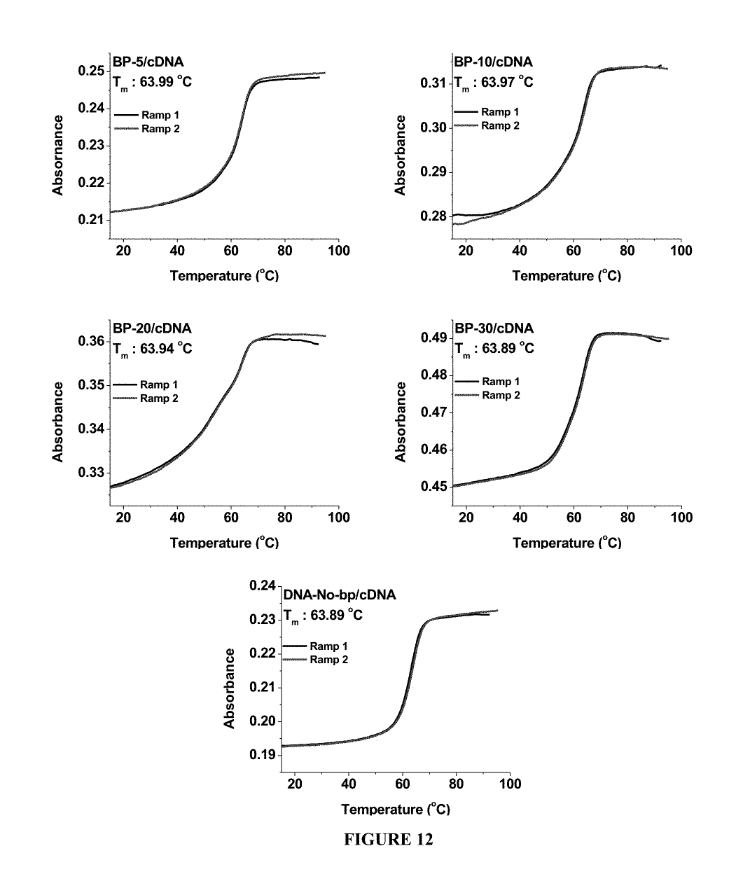

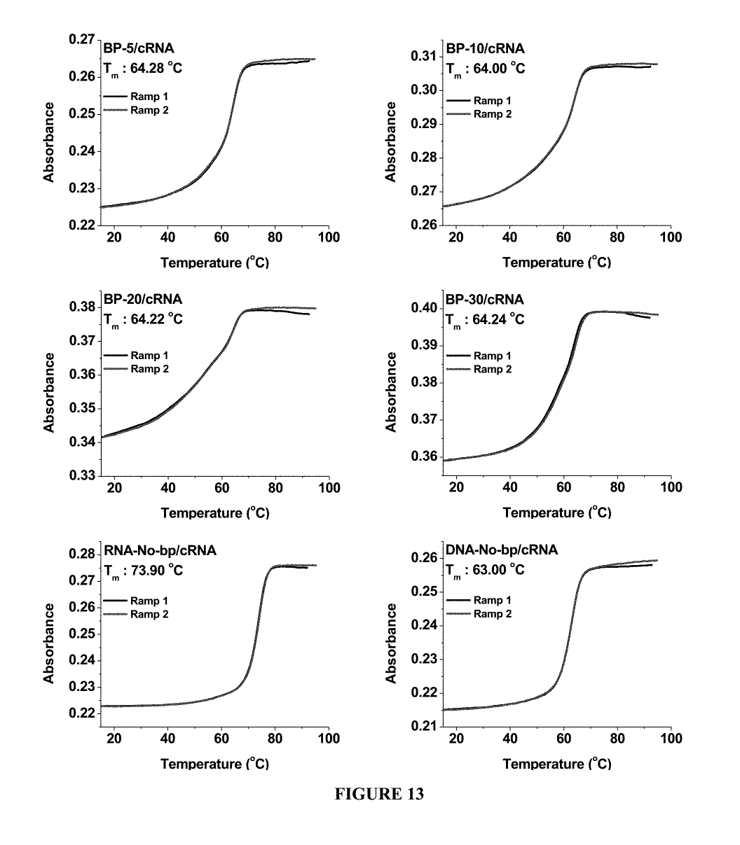

Melting temperature (Tₘ) of these probes with complementary DNA and RNA sequences were measured. Nearly identical Tₘ values of the BP probes compared to the unmodified DNA strand demonstrated that the presence of the BP containing signaling motif does not have any significant effect on probe's binding ability to the DNA and RNA target (FIGS. 12 and 13).

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A detection probe comprising a boranephosphonate probe moiety and a target selective moiety.

2. The detection probe of claim 1, wherein said detection probe comprises a plurality of said boranephosphonate probe moieties.

3. The detection probe of claim 1, wherein said target selective moiety comprises an oligonucleotide moiety.

4. The detection probe of claim 1, wherein said target selective moiety comprises a CRISPR-cas9 system having a small guide RNA oligomer (sgRNA) and a cas9 variant lacking active endonuclease domains (dcas9).

5. The detection probe of claim 1, wherein said target selective moiety comprises a DNA intercalator.

6. The detection probe of claim 1, wherein said boranephosphonate probe moiety is attached to said target selective moiety optionally through a linker.

7. The detection probe of claim 1, wherein said target selective moiety comprises a DNA intercalator.

8. The detection probe of claim 7, wherein said boranephosphonate probe moiety is linked to said DNA intercalator.

9. A method for detecting the presence or the location of a target molecule in a sample, said method comprising:
contacting a sample with a detection probe of claim 1 under conditions sufficient to form a probe-target complex when a target molecule is present in the sample;
reacting said boranephosphonate probe moiety with a metal ion solution under conditions sufficient to produce a metal nanoparticle (MNP); and
analyzing the MNP to determine the presence of or the location of said target molecule in said sample.

10. The method of claim 9, wherein said analysis of the MNP is conducted by visualization, using an electron microscopy, using a light microscopy, using a spectrometer, or a combination thereof.

11. The method of claim 9, wherein said target molecule is a nucleic acid sequence.

12. The method of claim 11, wherein said sample comprises a cell, a chromatin or a tissue section.

13. The method of claim 12, wherein said method is used to determine the location of a target nucleic acid sequence within the chromatin.

14. The method of claim 9, wherein said detection probe comprises a CRISP-cas9 system having a small guide RNA oligomer (sgRNA) and a cas9 variant lacking active endonuclease domains (dcas9).

15. The method of claim 14, wherein sgRNA comprises or is attached to said boranephosphonate probe moiety.

16. The method of claim 9, wherein said target selective moiety comprises a DNA intercalator.

17. The method of claim 16, wherein said boranephosphonate probe moiety is linked to said DNA intercalator.

18. The method of claim 9, wherein said target selective moiety is attached to a surface of a solid substrate that is capable of selectively binding to a portion of said target molecule to form a target-capture hybrid complex having a portion of said target molecule that is unbound to said target selective moiety when said target molecule is present in said sample, and wherein said boranephosphonate probe moiety is capable of binding to at least a portion of said unbound portion of said target molecule.

19. A detection probe comprising a boranephosphonate-pyridinium moiety and a target selective moiety.

20. The detection probe of claim 19, wherein said detection probe further comprises an oligonucleotide.


SEQUENCE LISTING (6 sequences)

SEQ ID NO: 1 - DNA, 45 bp, Artificial Sequence
  Capture Probe
  misc_feature (30)..(31): Boranephosphonate linkage
ttttttttttttttttcagtagggaggaaggtggttaagttaata

SEQ ID NO: 2 - DNA, 50 bp, Artificial Sequence
  Target Oligonucleotide
ggctccactaaatagacgcatattaacttaaccaccttcctccctactga

SEQ ID NO: 3 - DNA, 27 bp, Artificial Sequence
  BP-5 Probe Molecule
  misc_feature (21)..(26): Each nucleotide is linked with Boranephosphonate linkage
  misc_feature (26)..(27): Phosphorothioate linkage
tgcgtctatttagtggagccttttttt

SEQ ID NO: 4 - DNA, 32 bp, Artificial Sequence
  BP-10 Probe Molecule
  misc_feature (21)..(31): Each nucleotide is linked iwth boranephosphonate linkage
  misc_feature (31)..(32): Phosphorothioate linkage
tgcgtctatttagtggagcctttttttttttt

SEQ ID NO: 5 - DNA, 42 bp, Artificial Sequence
  BP-20 Probe Molecule
  misc_feature (21)..(41): Each nucleotide is linked with boranephosphonate linkage
  misc_feature (41)..(42): Nucleotides are linked via a phosphorothioate linkage
tgcgtctatttagtggagcctttttttttttttttttttttt

SEQ ID NO: 6 - DNA, 52 bp, Artificial Sequence
  BP-30 Probe Molecule
  misc_feature (21)..(51): Each nucleotide is linked via a boranephosphonate linkage
  misc_feature (51)..(52): Nucletoides are linked with a phosphorothioate linkage
tgcgtctatttagtggagcctttttttttttttttttttttttttttttttt